(19) United States
(12) Patent Application Publication
(10) Pub. No.: US 2025/0114019 A1
(43) Pub. Date: Apr. 10, 2025

(54) SYSTEMS AND METHODS FOR PERFORMANCE FATIGABILITY INDEX

(71) Applicant: UNIVERSITY OF PITTSBURGH-OF THE COMMONWEALTH SYSTEM OF HIGHER EDUCATION, Pittsburgh, PA (US)

(72) Inventors: Nancy W. GLYNN, Pittsburgh, PA (US); Yujia QIAO, Pittsburgh, PA (US)

(21) Appl. No.: 18/836,046

(22) PCT Filed: Feb. 9, 2023
(86) PCT No.: PCT/US2023/012679

§ 371 Date: Aug. 6, 2024

Related U.S. Application Data
(60) Provisional application No. 63/308,306, filed on Feb. 9, 2022.

(51) Int. Cl. A61B 5/11; A61B 5/00

(52) U.S. Cl. CPC A61B 5/112; A61B 5/6801; A61B 2562/0219

(57) ABSTRACT

Systems and methods for calculating performance fatigability measures for subjects based on raw accelerometry data are described herein. An example method includes receiving raw accelerometry data, where the raw accelerometry data is collected from a subject during a walking task. The method further includes analyzing the raw accelerometry data to calculate a performance fatigability index for the subject based on the raw accelerometry data. Optionally, the subject is an aging human adult.

### CROSS-REFERENCE TO RELATED APPLICATIONS

This application claims the benefit of U.S. provisional patent application No. 63/308,306, filed on Feb. 9, 2022, and titled “SYSTEMS AND METHODS FOR PERFORMANCE FATIGABILITY INDEX,” the disclosure of which is expressly incorporated herein by reference in its entirety.

### BACKGROUND

Fatigability is defined as one's vulnerability to fatigue anchored to a standardized physical task with specific duration and intensity. Greater fatigability, an early prognostic indicator of deleterious aging-related outcomes, is highly prevalent (30%-90%) in older adults, more common in women, and more severe with advanced age. It captures impending declines in physical functioning with great sensitivity and has been widely used as a standardized, valid, and reliable measure of an individual's vulnerability to fatigue in geriatric research. Two distinct constructs of fatigability have emerged: performance fatigability (i.e., performance deterioration) and perceived fatigability (i.e., perceived fatigue or exertion). Performance fatigability is quantified during a standardized physical task or activity, while perceived fatigability is measured via questionnaire or immediately following a standardized physical task. Many studies have shown that greater (i.e., more severe) perceived fatigability is associated with lower physical activity, higher chronic inflammation, greater cardiovascular burden, and predicts functional limitations, mobility decline, frailty, and mortality. However, little is known about performance fatigability among non-clinical populations of older adults due to limited objective, validated tools to measure performance fatigability.

It is therefore desirable to provide objective systems and methods to measure performance fatigability. Such systems and methods can facilitate efforts to study the underlying causes, prevention, and treatment of fatigue.

### SUMMARY

Systems and methods for calculating performance fatigability measures for subjects based on raw accelerometry data are described herein. Optionally, the subject is an aging human adult.

An example method includes receiving raw accelerometry data, the raw accelerometry data being collected from a subject during a walking task; and analyzing the raw accelerometry data to calculate a performance fatigability index for the subject based on the raw accelerometry data.

Additionally, the performance fatigability index for the subject represents a percentage of decrement in the subject's performance during the walking task. For example, the performance fatigability index for the subject may be a comparison between an area under an observed gait cadence-versus-time trajectory curve for the walking task and an area under a hypothetical gait cadence-versus-time trajectory curve for a walking task at maximal cadence.

Alternatively or additionally, the step of analyzing the raw accelerometry data includes obtaining an area under an observed gait cadence-versus-time trajectory curve for the walking task, where the performance fatigability index for the subject is calculated using the area under the observed gait cadence-versus-time trajectory curve for the walking task. Optionally, the step of obtaining the area under the observed gait cadence-versus-time trajectory curve for the walking task includes estimating a plurality of strides and a plurality of cadences for the walking task based on the raw accelerometry data; smoothing the cadences for the walking task to obtain a smoothed cadence trajectory; and determining the area under the observed gait cadence-versus-time trajectory curve for the walking task based on the smoothed cadence trajectory.

Alternatively or additionally, the step of analyzing the raw accelerometry data optionally includes partitioning the walking task into a plurality of segments; obtaining a first area under the observed gait cadence-versus-time trajectory curve for a first segment of the walking task; obtaining a second area under the observed gait cadence-versus-time trajectory curve for a second segment of the walking task; and assigning respective weights to each of the first and second areas under the observed gait cadence-versus-time trajectory curve. The performance fatigability index for the subject may be calculated using a sum of the weighted first and second areas under the observed gait cadence-versus-time trajectory curve.

Alternatively or additionally, the method optionally further includes collecting the raw accelerometry data from the subject while the subject completes the walking task.

Alternatively or additionally, the raw accelerometry data is received from a triaxial accelerometer. Optionally, the triaxial accelerometer is worn by the subject.

Alternatively or additionally, a length of the walking task can be selected to elicit a change in the subject's cadence during the walking task. For example, the length of the walking task may be at least 400 meters. Alternatively or additionally, the length of the walking task may be at least 4 minutes.

Alternatively or additionally, the walking task is a walk at a usual pace for the subject. Alternatively or additionally, the walking task is a walk at a fast pace for the subject.

Alternatively or additionally, the method optionally further includes classifying the subject into one of a plurality of physical performance decline categories based on the performance fatigability index.

An example system includes triaxial accelerometer and a computing device operably coupled to the accelerometer, the computing device including a processor and a memory operably coupled to the processor. The computing device is configured to receive raw accelerometry data from the triaxial accelerometer, and analyze the raw accelerometry data to calculate a performance fatigability index for a subject based on the raw accelerometry data.

Additionally, the performance fatigability index for the subject represents a percentage of decrement in the subject's performance during the walking task. For example, the performance fatigability index for the subject may be a comparison between an area under an observed gait cadence-versus-time trajectory curve for the walking task and an area under a hypothetical gait cadence-versus-time trajectory curve for a walking task at maximal cadence.

Alternatively or additionally, the step of analyzing the raw accelerometry data includes obtaining an area under an observed gait cadence-versus-time trajectory curve for the walking task, where the performance fatigability index for the subject is calculated using the area under the observed gait cadence-versus-time trajectory curve for the walking task. Optionally, the step of obtaining the area under the observed gait cadence-versus-time trajectory curve for the walking task includes estimating a plurality of strides and a plurality of cadences for the walking task based on the raw accelerometry data; smoothing the cadences for the walking task to obtain a smoothed cadence trajectory; and determining the area under the observed gait cadence-versus-time trajectory curve for the walking task based on the smoothed cadence trajectory.

Alternatively or additionally, the step of analyzing the raw accelerometry data optionally includes partitioning the walking task into a plurality of segments; obtaining a first area under the observed gait cadence-versus-time trajectory curve for a first segment of the walking task; obtaining a second area under the observed gait cadence-versus-time trajectory curve for a second segment of the walking task; and assigning respective weights to each of the first and second areas under the observed gait cadence-versus-time trajectory curve. The performance fatigability index for the subject may be calculated using a sum of the weighted first and second areas under the observed gait cadence-versus-time trajectory curve.

Alternatively or additionally, a length of the walking task can be selected to elicit a change in the subject's cadence during the walking task. For example, the length of the walking task may be at least 400 meters. Alternatively or additionally, the length of the walking task may be at least 4 minutes.

Alternatively or additionally, the walking task is a walk at a usual pace for the subject. Alternatively or additionally, the walking task is a walk at a fast pace for the subject.

Alternatively or additionally, the computing device is optionally further configured to classify the subject into one of a plurality of physical performance decline categories based on the performance fatigability index.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a block diagram of an example system for calculating performance fatigability measures for subjects based on raw accelerometry data according to implementations described herein.

FIG. 2 is an example computing device.

FIG. 3 is a flow chart illustrating example operations for calculating performance fatigability measures for subjects based on raw accelerometry data according to implementations described herein.

FIG. 4 illustrates example accelerometry data observed during a fast-paced 400 m walking task. The green line 402 (Y-axis), red line 404 (X-axis), and blue line 406 (Z-axis) represent the three axes from the ActiGraph. The dashed box 410 represents the identified 400 m walking task, lasting approximately 5.6 min.

FIGS. 5A-5B illustrate individual-level smoothed cadence-versus-time trajectories. FIG. 5A illustrates cadence trajectories for 59 participants who completed the fast-paced 400 m walk. FIG. 5B illustrates cadence trajectories for 56 participants who completed the usual-paced 400 m walk.

FIGS. 6A-6B illustrate examples of two different individual's PPFI score from the fast-paced 400 m walk. The blue triangles in the background represent raw cadence estimates; the black line 602 represents the individual-smoothed cadence trajectory; and the red circle 604 represents the maximum cadence. For the participant in FIG. 6A, the maximum cadence was at the beginning of the walk, and the total time to complete the walk was 6.75 minutes; for the participant in FIG. 6B, the maximum cadence was at ˜1 minutes, and the total time to complete the walk was 5.5 minutes. A=dashed green area, B=shaded green area, C=dashed orange area, D=shaded orange area. The PPFI equation is: [(A/A+B)*(B/B+D)+(C/C+D)*(D/B+D)]*100%.

FIGS. 7A-7D illustrate distributions of the Pittsburgh Performance Fatigability Scale (PPFI) scores in the cross-sectional study described in the Examples below. FIG. 7A is a distribution of PPFI scores for the fast-paced 400 m walk (n=59); FIG. 7B is a distribution of PPFI scores for the usual-paced 400 m walk (n=56); FIG. 7C are distributions of PPFI scores for the fast-paced 400 m walk stratified by sex (n=33 women; n=26 men); FIG. 7D are distributions of PPFI scores for the usual-paced 400 m walk stratified by sex (n=33 women; n=23 men).

FIG. 8 illustrates Pearson correlations from the fast-paced 400 m walk between PPFI scores and age, physical performance, aerobic fitness, muscle power, and other perceived and performance fatigability measures (n=59).

FIG. 9 is a table showing Pearson correlations between PPFI scores from the fast-paced walk and age, physical performance, aerobic fitness, muscle power, and perceived and performance fatigability measures (n=59) stratified by physical function.

FIG. 10 illustrates Pearson correlations from the usual-paced walk between PPFI scores and physical performance, mobility, muscle power, and other perceived and performance fatigability measures (n=56).

FIG. 11 is a table showing Pearson correlations between PPFI scores from the usual-paced walk and age, physical performance, mobility, muscle power, and perceived and performance fatigability measures (n=56) stratified by physical function.

FIG. 12 is a table showing associations of PPFI scores with physical performance, aerobic fitness, and muscle power.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” an, “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “about” or “approximately” when referring to a measurable value such as an amount, a percentage, and the like, is meant to encompass variations of ±20%, ±10%, ±5%, or ±1% from the measurable value.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

### Example System

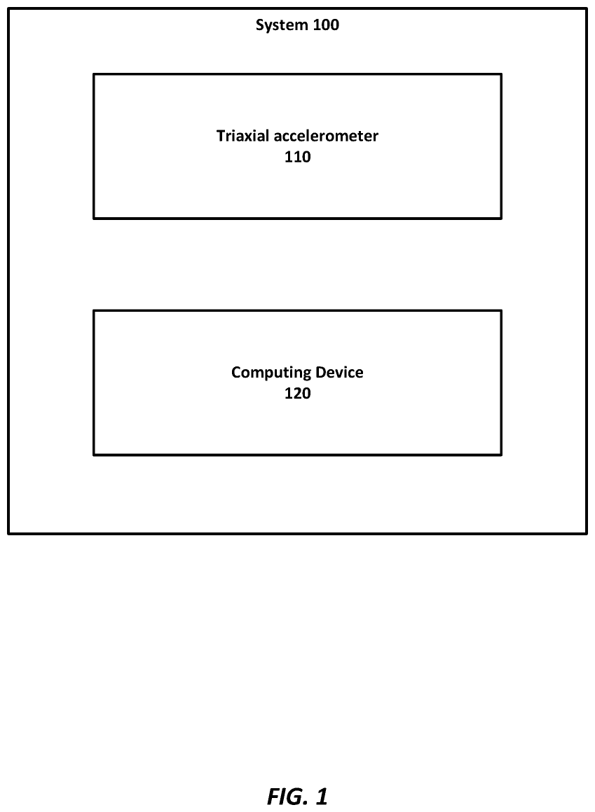

Referring now to FIG. 1, an example system for calculating performance fatigability measures for subjects based on raw accelerometry data is shown. The system 100 includes a triaxial accelerometer 110 and a computing device 120. The triaxial accelerometer 110 is configured to measure a subject's movements. The triaxial accelerometer 110 is configured for making measurements simultaneously in three orthogonal axes. Triaxial accelerometers are known in the art. For example, the GT3X+ activity monitor from The ActiGraph LLC of Pensacola, FL is a known triaxial accelerometer. It should be understood that the GT3X+ activity monitor is provided only as an example triaxial accelerometer. This disclosure contemplates using a triaxial accelerometer other than the GT3X+ activity monitor. In some implementations, the system 100 includes a single triaxial accelerometer 110. In some implementations, the system 100 includes a plurality of triaxial accelerometers 110. Optionally, the triaxial accelerometer 110 is worn by the subject, for example, while the subject completes a walking task (described in further detail below). In some implementations, the triaxial accelerometer 110 is a wrist-worn device. It should be understood that a wrist-worn triaxial accelerometer is provided only as an example. This disclosure contemplates that the triaxial accelerometer 110 may be worn on the subject's arm, ankle, hip, or other body part.

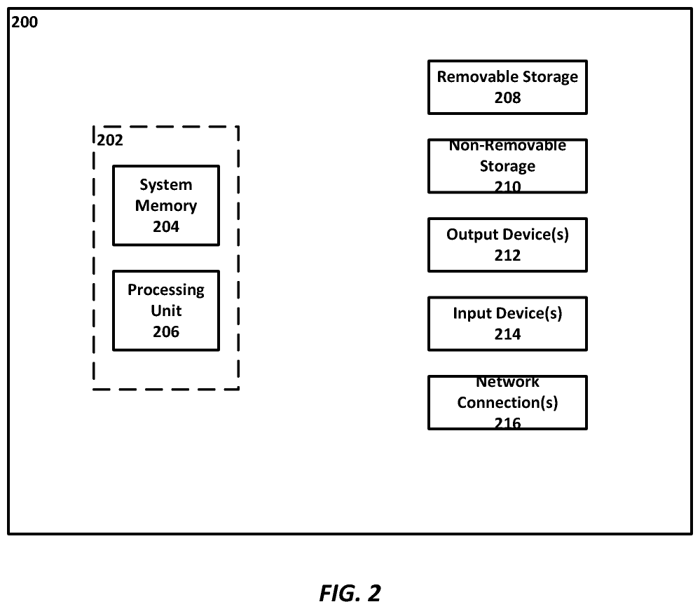

Additionally, the computing device 120 includes at least one processing unit and memory (e.g., the basic computing block shown by dashed line 202 in FIG. 2). Optionally, the computing device 120 is a computing device such as that shown in FIG. 2. This disclosure contemplates that the computing device 120 may be a smartphone, laptop, desktop, or tablet computer. The triaxial accelerometer 110 and computing device 120 discussed above can be coupled through one or more communication links. This disclosure contemplates the communication links are any suitable communication link. For example, a communication link may be implemented by any medium that facilitates data exchange between the triaxial accelerometer 110 and computing device 120 including, but not limited to, wired, wireless and optical links. Optionally, the triaxial accelerometer 110 and computing device 120 can be connected by one or more networks. This disclosure contemplates that the networks are any suitable communication network. The networks can be similar to each other in one or more respects. Alternatively or additionally, the networks can be different from each other in one or more respects. The networks can include a local area network (LAN), a wireless local area network (WLAN), a wide area network (WAN), a metropolitan area network (MAN), a virtual private network (VPN), etc., including portions or combinations of any of the above networks.

### Example Computing Device

It should be appreciated that the logical operations described herein with respect to the various figures may be implemented (1) as a sequence of computer implemented acts or program modules (i.e., software) running on a computing device (e.g., the computing device described in FIG. 2), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 2, an example computing device 200 upon which the methods described herein may be implemented is illustrated. It should be understood that the example computing device 200 is only one example of a suitable computing environment upon which the methods described herein may be implemented. Optionally, the computing device 200 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 200 typically includes at least one processing unit 206 and system memory 204. Depending on the exact configuration and type of computing device, system memory 204 may be volatile (such as random access memory (RAM)), non-volatile (such as read-only memory (ROM), flash memory, etc.), or some combination of the two. This most basic configuration is illustrated in FIG. 2 by dashed line 202. The processing unit 206 may be a standard programmable processor that performs arithmetic and logic operations necessary for operation of the computing device 200. The computing device 200 may also include a bus or other communication mechanism for communicating information among various components of the computing device 200.

Computing device 200 may have additional features/functionality. For example, computing device 200 may include additional storage such as removable storage 208 and non-removable storage 210 including, but not limited to, magnetic or optical disks or tapes. Computing device 200 may also contain network connection(s) 216 that allow the device to communicate with other devices. Computing device 200 may also have input device(s) 214 such as a keyboard, mouse, touch screen, etc. Output device(s) 212 such as a display, speakers, printer, etc. may also be included. The additional devices may be connected to the bus in order to facilitate communication of data among the components of the computing device 200. All these devices are well known in the art and need not be discussed at length here.

The processing unit 206 may be configured to execute program code encoded in tangible, computer-readable media. Tangible, computer-readable media refers to any media that is capable of providing data that causes the computing device 200 (i.e., a machine) to operate in a particular fashion. Various computer-readable media may be utilized to provide instructions to the processing unit 206 for execution. Example tangible, computer-readable media may include, but is not limited to, volatile media, non-volatile media, removable media and non-removable media implemented in any method or technology for storage of information such as computer readable instructions, data structures, program modules or other data. System memory 204, removable storage 208, and non-removable storage 210 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 206 may execute program code stored in the system memory 204. For example, the bus may carry data to the system memory 204, from which the processing unit 206 receives and executes instructions. The data received by the system memory 204 may optionally be stored on the removable storage 208 or the non-removable storage 210 before or after execution by the processing unit 206.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Example Methods

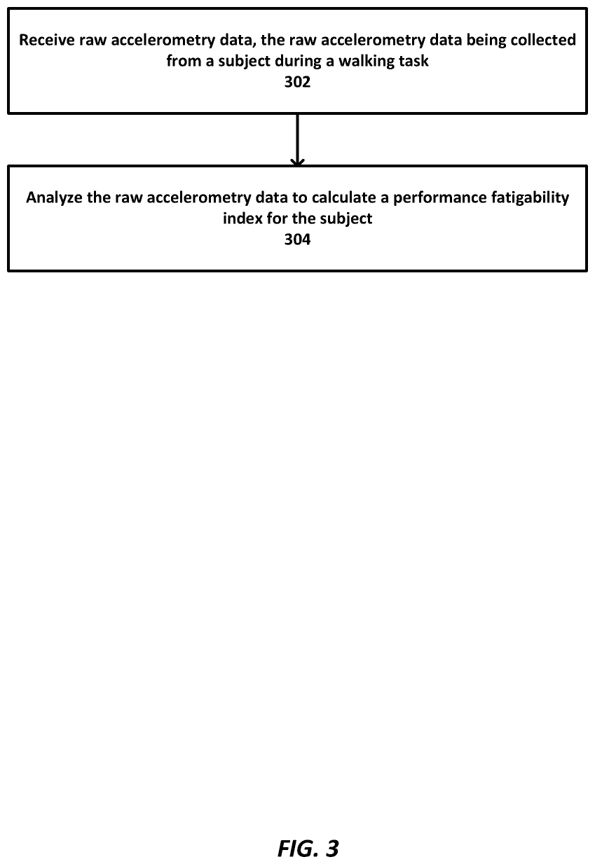

Referring now to FIG. 3, example operations for calculating performance fatigability measures for subjects based on raw accelerometry data are shown. This disclosure contemplates that the operations of FIG. 3 can optionally be performed using the system described above with respect to FIG. 1. As described herein, efforts to study performance fatigability have been limited due to conventional technology limitations. Accelerometry and advanced statistical methods have facilitated the ability to quantify performance fatigability more granularly via objective detection of performance decline. Accordingly, the systems and methods described herein have been developed using accelerometry data in order to address limitations of conventional technologies. Such limitations include, but are not limited to, assumptions of walking speed and/cadence declining consistently or linearly during a walking task, insufficient walking patterns captured, lack of comparability across walking tasks, and undue influence of the motivation effect. The systems and methods described herein overcome such technical challenges by analyzing the whole spectrum of cadence trajectory, smoothing cadence trajectories in an individualized manner, providing a metric that can be applied to different walking tasks, and weighting calculations to minimize the impact of the motivation effect. As a result, the systems and methods described herein provide for an objective and sensitive means to calculate performance fatigability measures.

At step 302, raw accelerometry data is received, for example by a computing device (e.g., computing device 120 of FIG. 1). The raw accelerometry data can be collected from a subject during a walking task. Optionally, the raw accelerometry data is collected using a triaxial accelerometer (e.g., triaxial accelerometer 110 of FIG. 1). Optionally, the subject has a wearable triaxial accelerometer for use during the walking task. In the implementations described herein, the subject is a human. Optionally, the subject is an aging human. Additionally, the walking task described herein is selected to elicit a change in the subject's cadence during the walking task. In some implementations, the walking task is a walk at a usual pace for the subject. In other implementations, the walking task is a walk at a fast pace for the subject. It should be understood that the length of such walking task will vary depending on the state and/or health of the subject. Subjects in a worse state and/or health may experience change in cadence more quickly than subjects in a better state and/or health. Alternatively or additionally, the length of such walking task will vary depending on the pace (e.g., normal or fast). In some implementations, the length of the walking task may be at least 400 meters. Alternatively or additionally, in some implementations, the length of the walking task may be 5-7 minutes, optionally at least 4 minutes. A walking task of at least 400 meter or at least 4 minutes has been found to be of sufficient length to elicit a change in cadence for a typical aging human subject. It should be understood that at least 400 meter or at least 4 minutes are provided only as example lengths for the walking task. Accordingly, in other implementations, this disclosure contemplates that the length of the walking task may be less than 400 meters (e.g., 100 meters, 200 meters, 300 meters, etc.) or greater than 400 meters (e.g., 500 meters, 600 meters, 700 meters, etc.), or that the length of the walking task may be less than 4 minutes (e.g., 2 minutes, 3 minutes, etc.) or greater than 4 minutes (e.g., 5 minutes, 6 minutes, 7 minutes, etc.). This is particularly the case when the subject is not a typical aging human, e.g., a human of worse and/or better state and/or health than the average aging human.

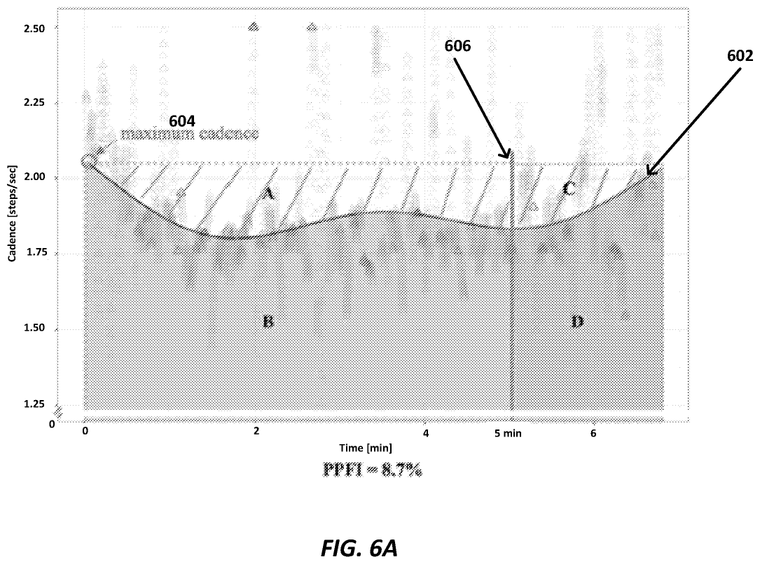

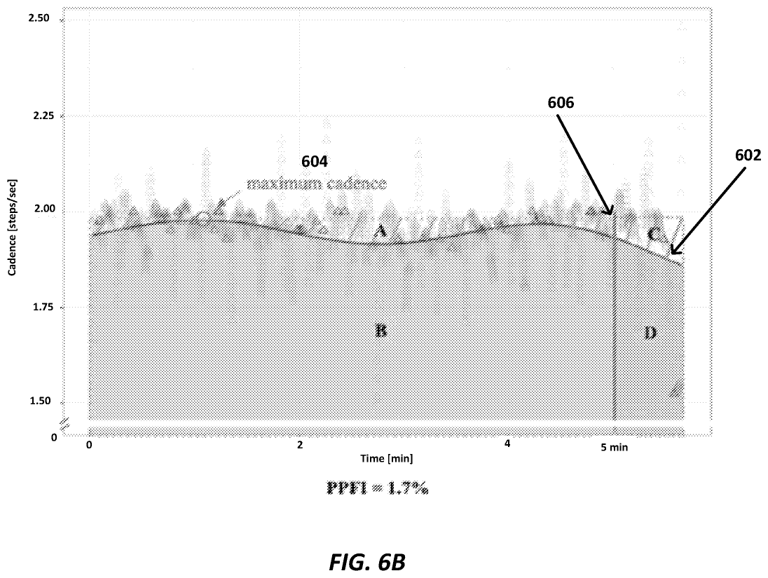

At step 304, the raw accelerometry data is analyzed, for example by a computing device (e.g., computing device 120 of FIG. 1), to calculate a performance fatigability index for the subject. The performance fatigability index for the subject represents a percentage of decrement in the subject's performance during the walking task. For example, the performance fatigability index for the subject may be a comparison between an area under an observed gait cadence-versus-time trajectory curve for the walking task (e.g., areas B and D in FIGS. 6A-6B) and an area under a hypothetical gait cadence-versus-time trajectory curve for a walking task at maximal cadence (e.g., areas A, B, C and D in FIGS. 6A-6B).

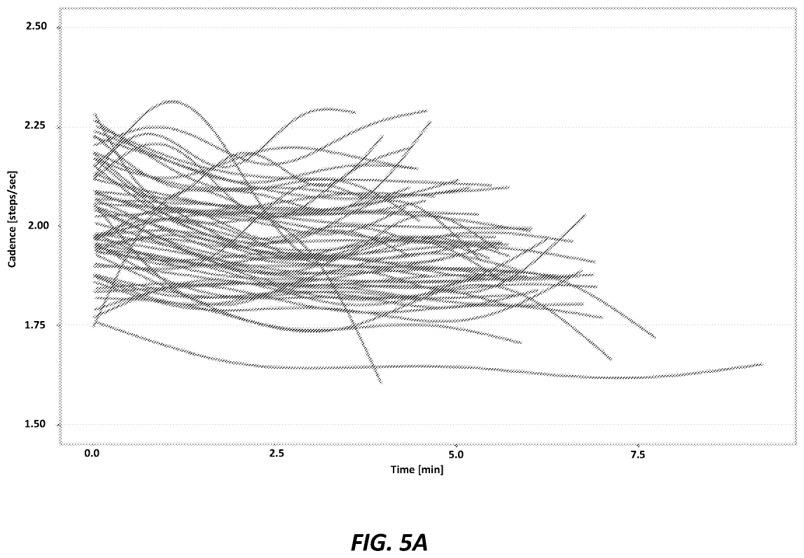

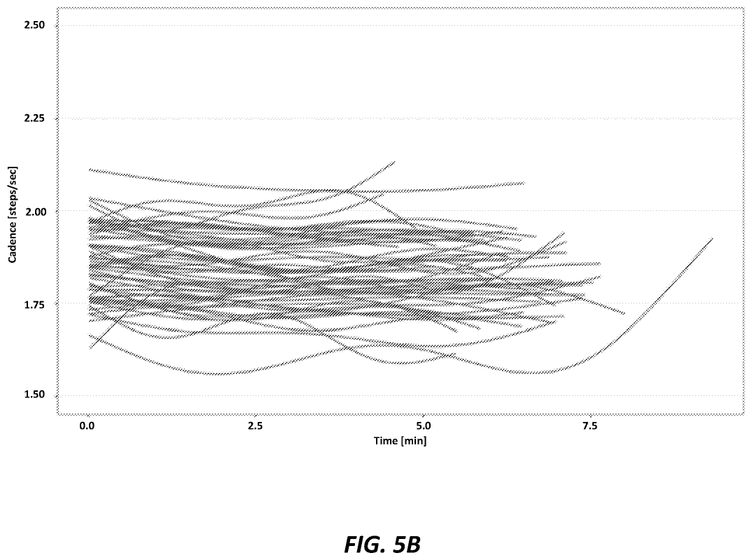

In some implementations, the step of analyzing the raw accelerometry data includes obtaining an area under an observed gait cadence-versus-time trajectory curve for the walking task, as well as obtaining an area under a hypothetical gait cadence-versus-time trajectory curve for a walking task at maximal cadence. Optionally, the method may include estimating a plurality of strides and a plurality of cadences for the walking task based on the raw accelerometry data. As described in Examples below, a stride is a full step cycle and cadence is a rate of step cycles. Strides and cadence can be estimated by analyzing the raw accelerometry data, for example, using the Adaptive Empirical Pattern Transformation (ADEPT) R package. In the examples herein, cadence is estimated once per second. It should be understood that the estimation period for cadence is provided only as an example. This disclosure contemplates that the estimation period for cadence may be greater or less than 1 second depending on the length of identified stride. Additionally, it should be understood that cadence may vary over the walking task. It should be understood that the ADEPT R package is provided only as an example software tool for analyzing the raw accelerometry data to estimate strides and cadence. This disclosure contemplates using any known software tool for analyzing the raw accelerometry data to estimate strides and cadence. Thereafter, the method may further includes smoothing the cadences for the walking task to obtain a smoothed cadence trajectory. An example smoothed cadence trajectory curve is represented by reference number 602 in FIGS. 6A-6B. A smoothing window can be used to obtain the smoothed cadence trajectory. Alternatively or additionally, a parametric regression technique such as spline regression can optionally be used to obtain the smoothed cadence trajectory. This disclosure contemplates that the parametric regression can be performed to individualize the smoothed cadence trajectory. Example smoothed cadence-versus-time trajectories are illustrated in FIGS. 5A-5B. Additionally, example techniques for smoothing window and parametric regression are described below in the Examples. It should be understood that the techniques described in the Examples are non-limiting examples. Thereafter, the method may further include determining the area under the observed gait cadence-versus-time trajectory curve for the walking task based on the smoothed cadence trajectory (e.g., areas B+D, i.e., the area under the curve 602, in FIGS. 6A-6B). The method may further include determining the area under the hypothetical gait cadence-versus-time trajectory curve for the walking task at maximal cadence (e.g., areas A+B+C+D in FIGS. 6A-6B). The areas described above can be determined, for example, using “DescTools” R package with the default trapezoid method. It should be understood that the “DescTools” R package is provided only as an example software tool for determining the areas described above. This disclosure contemplates using any known software tool for determining the areas described above. Thereafter, the performance fatigability index for the subject is calculated, for example, as a ratio of an area under an observed gait cadence-versus-time trajectory curve for the walking task to an area under a hypothetical gait cadence-versus-time trajectory curve for a walking task at maximal cadence. Such a ratio can be calculated according to the equations used in FIGS. 6A-6B, for example. Alternatively, the performance fatigability index for the subject can be calculated using Equation (1) in the Examples.

Optionally, in some implementations, the method may include partitioning the walking task into a plurality of segments and obtaining respective areas under an observed gait cadence-versus-time trajectory curve for the segments of the walking task. For example, the walking task can optionally be partitioned into a plurality of segments based on population average gait speed. As used herein, population average gait speed includes the time that most participants needed to complete the walking task, while still leaving out on average at least 30 seconds at the end of the walk when participants usually sped up. Partitioning the walking task into a plurality of segments assists in minimizing the influence of the motivation effect (e.g., where the subject speeds up at the end of the task). In this implementation, the method may include estimating a plurality of strides and a plurality of cadences for the walking task based on the raw accelerometry data, and smoothing the cadences for the walking task to obtain a smoothed cadence trajectory. An example smoothed cadence trajectory curve is represented by reference number 602 in FIGS. 6A-6B. These techniques can be performed as described above. Thereafter, the method may include determining a first area under the observed gait cadence-versus-time trajectory curve for a first segment of the walking task (e.g., area B, i.e., the area under the curve 602 to the left of line 606, in FIGS. 6A-6B), and determining a second area under the observed gait cadence-versus-time trajectory curve for a second segment of the walking task (e.g., area D, i.e., the area under the curve 602 to the right of line 606, in FIGS. 6A-6B). The first and second areas can be summed (and optionally weighted as described below) to obtain the to obtain the area under the observed gait cadence-versus-time trajectory curve for the entire walking task. For example, the first segment of the walking task can be the first 5 minutes for a fast-paced walking task or the first 6 minutes for a usual-paced walking task as described in the Examples below. The second segment of the walking task can be the remaining portion of the walking task to completion. It should be understood that the 5 and 6 minute partitioning thresholds are provided only as examples and may have other values. For example, the first and second segments are partitioned by line 606 in FIGS. 6A-6B. The method may further include determining a third area under a hypothetical gait cadence-versus-time trajectory curve for the first segment of the walking task at maximal cadence (e.g., areas A+B in FIGS. 6A-6B), and determining a fourth area under a hypothetical gait cadence-versus-time trajectory curve for the second segment of the walking task at maximal cadence (e.g., areas C+D in FIGS. 6A-6B). The first, second, third, and fourth areas can be summed (and optionally weighted as described below) to obtain the to obtain the area under the hypothetical gait cadence-versus-time trajectory curve for the entire walking task at maximum cadence. This disclosure contemplates using any known software tool for determining the areas as described above. Additionally, different weights are assigned to each of the first and second areas under the observed gait cadence-versus-time trajectory curve. Weights can be assigned before summing the first and second areas. The weights can be selected to minimize the influence of motivation effect at the end of the walk and/or emphasize that the performance decrement occurred at the beginning of the walk. Thereafter, the performance fatigability index for the subject is calculated, for example, as a ratio of an area under an observed gait cadence-versus-time trajectory curve for the walking task to an area under a hypothetical gait cadence-versus-time trajectory curve for a walking task at maximal cadence. Such a ratio can be calculated according to the equations used in FIGS. 6A-6B, for example. Alternatively, the performance fatigability index for the subject can be calculated using Equations (2) and (3) in the Examples. Equations (2) and (3) represent calculations for the first and second segments, respectively.

Optionally, the subject can be classified into one of a plurality of physical performance decline categories based on the performance fatigability index, which is calculated as described above. For example, the categories may include, but are not limited to, no performance fatigability, moderate performance fatigability, and severe performance fatigability. It should be understood that less or more (e.g., sub categories within the examples) may be used. PPFI cut-points for the categories can be obtained by analyzing a dataset to generate one or more cut-points in PPFI scores that discriminate gait speed. The Examples below include an example analysis for generating PPFI cut-points.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Fatigability is defined as one's vulnerability to fatigue anchored to a standardized physical task with specific duration and intensity (Eldadah B A. Fatigue and fatigability in older adults. PM R. 2010 May; 2(5):406-13; Schrack J A, Simonsick E M, Glynn N W. Fatigability: A prognostic indicator of phenotypic aging. J Gerontol A, Biol Sci Med Sci. 2020 Sep. 16; 75(9):e63-6). Greater fatigability, an early prognostic indicator of deleterious aging-related outcomes, is highly prevalent (30%-90%) in older adults, more common in women, and more severe with advanced age (Eldadah B A. Fatigue and fatigability in older adults. PM R. 2010 May; 2(5):406-13; SchrackJA, Simonsick E M, Glynn N W. Fatigability: A prognostic indicator of phenotypic aging. J Gerontol A, Biol Sci Med Sci. 2020 Sep. 16; 75(9):e63-6), which captures impending declines in physical functioning with great sensitivity (Schrack J A, Simonsick E M, Glynn N W. Fatigability: A prognostic indicator of phenotypic aging. J Gerontol A, Biol Sci Med Sci. 2020 Sep. 16; 75(9):e63-6; Simonsick E M, Glynn N W, Jerome G J, Shardell M, Schrack J A, Ferrucci L. Fatigued, but Not Frail: Perceived Fatigability as a Marker of Impending Decline in Mobility-Intact Older Adults. J Am Geriatr Soc. 2016 Jun. 2; 64(6):1287-92; Simonsick E M, Schrack J A, Santanasto A J, Studenski S A, Ferrucci L, Glynn N W. Pittsburgh Fatigability Scale: One-Page Predictor of Mobility Decline in Mobility-Intact Older Adults. J Am Geriatr Soc. 2018 November; 66(11):2092-6) and has been widely used as a standardized, valid and reliable measure of an individual's vulnerability to fatigue in geriatric research. Two distinct constructs of fatigability have emerged: performance fatigability (i.e., performance deterioration) and perceived fatigability (i.e., perceived fatigue or exertion) (Eldadah B A. Fatigue and fatigability in older adults. PM R. 2010 May; 2(5):406-13; Schrack J A, Simonsick E M, Glynn N W. Fatigability: A prognostic indicator of phenotypic aging. J Gerontol A, Biol Sci Med Sci. 2020 Sep. 16; 75(9):e63-6). Performance fatigability is quantified during a standardized physical task or activity, while perceived fatigability is measured via questionnaire or immediately following a standardized physical task. Many studies have shown that greater (i.e., more severe) perceived fatigability is associated with lower physical activity (Wanigatunga A A, Simonsick E M, Zipunnikov V, Spira A P, Studenski S, Ferrucci L, et al. Perceived Fatigability and Objective Physical Activity in Mid- to Late-Life. J Gerontol A, Biol Sci Med Sci. 2018 Apr. 17; 73(5):630-5; Qiao Y S, Gmelin T, Renner S W, Boudreau R M, Martin S, Wojczynski M K, et al. Evaluation of the bidirectional relations of perceived physical fatigability and physical activity on slower gait speed. J Gerontol A, Biol Sci Med Sci. 2021 Sep. 13; 76(10):e237-44), higher chronic inflammation (Wanigatunga A A, Varadhan R, Simonsick E M, Carlson O D, Studenski S, Ferrucci L, et al. Longitudinal Relationship Between Interleukin-6 and Perceived Fatigability Among Well-Functioning Adults in Mid-to-Late Life. J Gerontol A, Biol Sci Med Sci. 2019 Apr. 23; 74(5):720-5; Cooper R, Popham M, Santanasto A J, Hardy R, Glynn N W, Kuh D. Are BMI and inflammatory markers independently associated with physical fatigability in old age?Int J Obes. 2019; 43(4):832-41), greater cardiovascular burden (Qiao Y, Martinez-Amezcua P, Wanigatunga A A, Urbanek J K, Simonsick E M, Ferrucci L, et al. Association Between Cardiovascular Risk and Perceived Fatigability in Mid-to-Late Life. J Am Heart Assoc. 2019 Aug. 20; 8(16):e013049), and predicts functional limitations, mobility decline (Simonsick E M, Glynn N W, Jerome G J, Shardell M, Schrack J A, Ferrucci L. Fatigued, but Not Frail: Perceived Fatigability as a Marker of Impending Decline in Mobility-Intact Older Adults. J Am Geriatr Soc. 2016 Jun. 2; 64(6):1287-92; Simonsick E M, Schrack J A, Santanasto A J, Studenski S A, Ferrucci L, Glynn N W. Pittsburgh Fatigability Scale: One-Page Predictor of Mobility Decline in Mobility-Intact Older Adults. J Am Geriatr Soc. 2018 November; 66(11):2092-6; Gresham G, Dy S M, Zipunnikov V, Browner I S, Studenski S A, Simonsick E M, et al. Fatigability and endurance performance in cancer survivors: Analyses from the Baltimore Longitudinal Study of Aging. Cancer. 2018 Mar. 15; 124(6):1279-87), frailty (Schnelle J F, Buchowski M S, Ikizler T A, Durkin D W, Beuscher L, Simmons SF. Evaluation of two fatigability severity measures in elderly adults. J Am Geriatr Soc. 2012 Aug. 2; 60(8):1527-33), and mortality (Glynn N W, Gmelin T, Renner S W, QiaoScM YS, Boudreau R M, Feitosa M F, et al. Perceived Physical Fatigability Predicts All-Cause Mortality in Older Adults. J Gerontol A, Biol Sci Med Sci. 2021 Dec. 15). However, little is known about performance fatigability among non-clinical populations of older adults due to limited objective, validated tools to measure performance fatigability.

A performance fatigability measure for older adults, called Performance Deterioration, defined as the percent of gait speed slowing between the 2^{nd} and 9^{th} (out of 10) laps during a fast-paced 400 m long-distance corridor walk was previously developed (Simonsick E M, Schrack J A, Glynn N W, Ferrucci L. Assessing fatigability in mobility-intact older adults. J Am Geriatr Soc. 2014 February; 62(2):347-51). Existing performance fatigability measures developed by others also used similar computational approaches by comparing gait speeds at the end and the beginning of an in-lab walking task (Van Geel F, Moumdjian L, Lamers I, Bielen H, Feys P. Measuring walking-related performance fatigability in clinical practice: a systematic review. Eur J Phys Rehabil Med. 2020 February; 56(1):88-103). Despite wide use, these measures have limited utility for identifying performance fatigability in younger and/or higher-functioning persons (Glynn N W, Qiao Y S, Simonsick E M, Schrack J A. Response to “comment on: fatigability: A prognostic indicator of phenotypic aging”. J Gerontol A, Biol Sci Med Sci. 2021 Jul. 13; 76(8):e161-2). A key assumption that walking speed would decline constantly and linearly over a particular walking task as persons experienced fatigue, does not consistently occur. It has been subsequently demonstrated that gait speed often fluctuates during walking-related tasks in older adults (Glynn N W, Qiao Y S, Simonsick E M, Schrack J A. Response to “comment on: fatigability: A prognostic indicator of phenotypic aging”. J Gerontol A, Biol Sci Med Sci. 2021 Jul. 13; 76(8):e161-2; Lindemann U, Krumpoch S, Becker C, Sieber C C, Freiberger E. The course of gait speed during a 400 m walk test of mobility limitations in community-dwelling older adults. Z Gerontol Geriatr. 2021 Jun. 11). Specifically, older adults tend to slow down in the middle of the walking task and speed up at the end, potentially because they are motivated to finish. Second, only a limited snapshot of information (e.g., lap intervals and gait speed) was used to develop Performance Deterioration and other existing performance fatigability measures. Fortunately, advances in technology and statistical methods over the past ten years has expanded the utility of wearable accelerometers to continuously and granularly monitor detailed aspects of walking patterns during standardized tasks. Thus, it is now feasible to develop a performance fatigability measure that utilizes the entire range of gait characteristics during walking tasks to capture situations where participants' performance may exhibit decline at any time during a walking task or decline minimally, thus reducing the potential for misclassification. Third, each performance fatigability measure utilizes different types of walking tasks, resulting in different scales and distributions of fatigability scores making it is difficult to compare across studies and measures. To this end, developing an objective and sensitive performance fatigability measure is needed to better understand fatigability's role in the aging process.

To develop a new performance fatigability measure, the concept of motor fatigue and fatigability often used in the multiple sclerosis literature (Schwid S R, Thornton C A, Pandya S, Manzur K L, Sanjak M, Petrie M D, et al. Quantitative assessment of motor fatigue and strength in MS. Neurology. 1999 Sep. 11; 53(4):743-5; Severijns D, Zijdewind I, Dalgas U, Lamers I, Lismont C, Feys P. The assessment of motor fatigability in persons with multiple sclerosis: A systematic review. Neurorehabil Neural Repair. 2017 May; 31(5):413-31) was drawn upon. Motor fatigue is an acute activity-induced reduction of force or power of a muscle, and in laboratory settings is typically quantified as the reduction in maximal strength or power, or time to failure executing a submaximal task (Enoka R M, Duchateau J. Translating fatigue to human performance. Med Sci Sports Exerc. 2016 November; 48(11):2228-38). In community-dwelling older adults, we are interested in an individual's vulnerability to fatigue, captured by the degree of performance decrement (i.e. slowing down) during a standardized walking task (e.g., 400 m walk) that is indicative of aerobic fitness and mobility (Simonsick E M, Montgomery P S, Newman A B, Bauer D C, Harris T. Measuring fitness in healthy older adults: the Health ABC Long Distance Corridor Walk. J Am Geriatr Soc. 2001 November; 49(11):1544-8; Simonsick E M, Newman A B, Visser M, Goodpaster B, Kritchevsky S B, Rubin S, et al. Mobility limitation in self-described well-functioning older adults: importance of endurance walk testing. J Gerontol A, Biol Sci Med Sci. 2008 August; 63(8):841-7), rather than acute physiological reactions of the involved muscles (Enoka R M, Duchateau J. Translating fatigue to human performance. Med Sci Sports Exerc. 2016 November; 48(11):2228-38). Thus, long-distance walking tasks, such as 400 m or 6-minute walks, are commonly used as standardized physical tasks to mimic real-world activity (Kim I, Hacker E, Ferrans C E, Horswill C, Park C, Kapella M. Evaluation of fatigability measurement: Integrative review. Geriatr Nurs. 2018; 39(1):39-47). To develop an objective and sensitive performance fatigability measure, we capitalized on wrist-worn accelerometry, which measures accelerations in three orthogonal axes to provide information on the amount, frequency, and duration of movement as well as gait parameters (Karas M, Bai J, Straczkiewicz M, Harezlak J, Glynn N W, Harris T, et al. Accelerometry data in health research: challenges and opportunities. Stat Biosci. 2019 July; 11(2):210-37). A systematic review found that gait characteristics such as cadence and variability (i.e., coefficients of variation) in those characteristics, have been associated with task-induced fatigue (Barbieri F A, Santos PCR dos, Lirani-Silva E, Vitório R, Gobbi LTB, van Dieen J H. Systematic review of the effects of fatigue on spatiotemporal gait parameters. J Back Musculoskelet Rehabil. 2013; 26(2):125-3). Although mean value and variability-related gait parameters seemed promising candidates for measuring performance fatigability, these aggregated parameters captured less information about an individual's physical performance than a continuous gait trajectory. Additionally, these parameters were only reported in two studies with small sample sizes (<30 participants) using complex and non-portable multi-sensor measurements (Helbostad J L, Leirfall S, Moe-Nilssen R, Sletvold O. Physical fatigue affects gait characteristics in older persons. J Gerontol A, Biol Sci Med Sci. 2007 September; 62(9):1010-5; Yoshino K, Motoshige T, Araki T, Matsuoka K. Effect of prolonged free-walking fatigue on gait and physiological rhythm. J Biomech. 2004 August; 37(8):1271-80). Wrist-worn accelerometers have proven to be more comfortable to wear and validly track physical activity in older adults (Liu F, Wanigatunga A A, Schrack J A. Assessment of Physical Activity in Adults using Wrist Accelerometers. Epidemiol Rev. 2021 Jul. 2). Researchers have widely used wrist-worn accelerometers in the lab and free-living environments. Further, it is easier and more accessible to implement wrist-worn accelerometers into the general population and larger research studies, with greater compliance and wear time (Doherty A, Jackson D, Hammerla N, Plotz T, Olivier P, Granat M H, et al. Large scale population assessment of physical activity using wrist worn accelerometers: the UK biobank study. PLoS One. 2017 Feb. 1; 12(2):e0169649; Freedson P S, John D. Comment on “estimating activity and sedentary behavior from an accelerometer on the hip and wrist”. Med Sci Sports Exerc. 2013 May; 45(5):962-3). Therefore, previous work was built upon to develop an objective performance fatigability measure in older adults using wrist-worn accelerometry, to establish an easy-to-implement and sensitive performance fatigability measure for future epidemiological studies and clinical practice. The objective performance fatigability measure will allow us to more fully understand the health impact of greater or increasing fatigability and its role in the disablement pathway.

Study objectives include: 1) developing an accelerometry-based performance fatigability index, named the Pittsburgh Performance Fatigability Index (PPFI), appropriate for community-dwelling older adults, and 2) validating PPFI's associations with age, clinically relevant measures (including physical performance, aerobic fitness, mobility, muscle power), and existing fatigability measures.

### Example 1

### Methods

### Study Sample

The cross-sectional study conducted at the University of Pittsburgh in 2010 enrolled 68 (39 women and 29 men) community-dwelling older adults (age≥70 years) with a range of self-reported function using the Pittsburgh Claude D. Pepper Older Americans Independence Center Research Registry (30). Exclusion criteria included any self-reported health contraindication to physical testing (i.e., hip fracture, stroke in the past 12 months, cerebral hemorrhage in the past 6 months, heart attack, angioplasty, heart surgery in the past 3 months) or the inability to perform basic mobility tasks (i.e., chest pain during walking in the past 30 days, current treatment for shortness of breath or a lung condition, usual aching, stiffness, or pain in their lower limbs and joints, and bilateral difficulty in bending or straightening the knees fully). The current study included data from 64 participants that completed either a fast- or usual-paced 400 m walk and had valid accelerometry data. To maximize the sample size, we used all participants with valid ActiGraph data and completed fast-paced 400 m walk (n=59) or usual-paced 400 m walk (n=56) for their respective analyses.

### Fast- and Usual-Paced Walks

The walking course was located in an unobstructed, dedicated long corridor with traffic cones on both ends. During the first clinic visit, participants completed the fast-paced 400 m walk test with the instructions of completing the distance “as quickly as possible, without running, at a pace they could maintain” for ten laps (20-meter each direction) (30). During the second clinic visit, 8 to 14 days later, participants completed the usual-paced 400 m walk on the same course and were instructed to complete the distance “at their usual pace without overexerting themselves” (Lange-Maia B S, Newman A B, Strotmeyer E S, Harris T B, Caserotti P, Glynn N W. Performance on fast- and usual-paced 400-m walk tests in older adults: are they comparable?Aging Clin Exp Res. 2015 June; 27(3):309-14). For both walks, start and stop time of walking tasks were written in 24-hour clock time on lab notes, and time to completion (or the time until participant discontinued the task) as well as split times for each lap were recorded in seconds using a stopwatch. A test was stopped if the participant's heart rate exceeded 170 bpm/minute or they reported chest pain, tightness or pressure in the chest, shortness of breath, leg pain or feeling faint, lightheaded or dizzy, or they requested to stop.

### Accelerometry Measurements

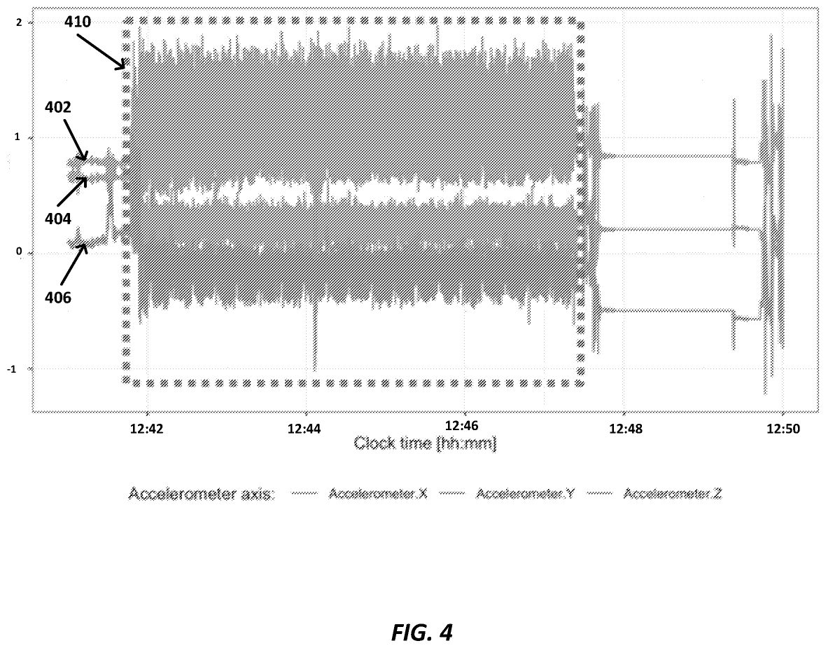

All participants wore an ActiGraph GT3X+ accelerometer (The ActiGraph LLC, Pensacola, FL) on the non-dominant wrist during both walking tasks. Raw accelerometry data were collected at a sample frequency of 80 Hz and expressed in gravity (g) units in three orthogonal axes. Data quality was assessed via visual examination. The start/stop clock times from the 400 m walks were recorded and used as anchors to visually locate the 400 m walks on the raw tri-axial accelerations signal plots by trained research staff. Repetitive and periodic patterns observed on the raw acceleration signal plots represent the 400 m walk (FIG. 4) (Urbanek J K, Zipunnikov V, Harris T, Fadel W, Glynn N, Koster A, et al. Prediction of sustained harmonic walking in the free-living environment using raw accelerometry data. Physiol Meas. 2018 Feb. 28; 39(2):02NT02). The time differences between the start/stop clock times recorded at the time of the walks and the raw accelerometry data averaged 1 second (range: 0 second-13 second). Once the raw accelerometry data from each of the 400 m walk plots were segmented, they were saved for the derivation of PPFI, which is detailed in Example 2.

### Clinically Relevant Measures

The Short Physical Performance Battery (SPPB), a widely-used objective measure of physical function and associated with fatigability (LaSorda K R, Gmelin T, Kuipers A L, Boudreau R M, Santanasto A J, Christensen K, et al. Epidemiology of perceived physical fatigability in older adults: the Long Life Family Study. J Gerontol A, Biol Sci Med Sci. 2020 Sep. 16; 75(9):e81-8), was administered at the first clinic visit. SPPB consists of three components: 1) balance tests with side-by-side, semi-tandem and full-tandem positions, 2) a 6 m usual-paced walk, and 3) five repeated chair stands. Each component was scored 0 (unable to complete) to 4 (best) and a summary score was calculated ranging from 0-12, with higher scores indicating better physical function (Guralnik J M, Simonsick E M, Ferrucci L, Glynn R J, Berkman L F, Blazer D G, et al. A short physical performance battery assessing lower extremity function: Association with self-reported disability and prediction of mortality and nursing home admission. J Gerontol. 1994 March; 49(2):M85-94). The SPPB score was further categorized into better (≥10) vs. worse (<10) physical function (Pavasini R, Guralnik J, Brown J C, di Bari M, Cesari M, Landi F, et al. Short Physical Performance Battery and all-cause mortality: systematic review and meta-analysis. BMC Med. 2016 Dec. 22; 14(1):215). Given the ceiling effects of SPPB scores, the individual components of usual gait speed (m/s) and chair stands speed (stands/second) were separately evaluated as continuous measures for their associations with PPFI. For participants who did not attempt or complete the chair stands, time to completion was assigned at 60 seconds (Santanasto A J, Glynn N W, Lovato L C, Blair S N, Fielding R A, Gill T M, et al. Effect of Physical Activity versus Health Education on Physical Function, Grip Strength and Mobility. J Am Geriatr Soc. 2017 July; 65(7):1427-33), which equates to a speed of 0.083 stands/second. Balance tests were individually evaluated because this test does not yield a continuous measure.

Furthermore, aerobic fitness was measured as time to complete the fast-paced 400 m walking task (seconds). The shorter the time, the better aerobic fitness (Simonsick E M, Fan E, Fleg J L. Estimating cardiorespiratory fitness in well-functioning older adults: treadmill validation of the long distance corridor walk. J Am Geriatr Soc. 2006 January; 54(1):127-32). Mobility was measured as time to complete the usual-paced 400 m walking task (seconds). The shorter the time, the better mobility (Fielding R A, Rejeski W J, Blair S, Church T, Espeland M A, Gill T M, et al. The Lifestyle Interventions and Independence for Elders Study: design and methods. J Gerontol A, Biol Sci Med Sci. 2011 Nov. 1; 66(11):1226-37). Peak leg power was obtained as the highest measurement of power achieved from 40%-70% 1 Repetition Maximum using the Keiser pneumatic resistance device (A420 model; Keiser Sports Health Equipment, Fresno, CA) at the second clinic visit. The detailed protocol can be found elsewhere (Winger M E, Caserotti P, Ward R E, Boudreau R M, Hvid L G, Cauley J A, et al. Jump power, leg press power, leg strength and grip strength differentially associated with physical performance: The Developmental Epidemiologic Cohort Study (DECOS). Exp Gerontol. 2021 March; 145:111172).

Pittsburgh Fatigability Scale (PFS) was measured at the first clinic visit, the PFS is a validated, self-administered 10-item tool for older adults (Glynn N W, Santanasto A J, Simonsick E M, Boudreau R M, Beach S R, Schulz R, et al. The Pittsburgh Fatigability Scale for older adults: development and validation. J Am Geriatr Soc. 2015 January; 63(1):130-5). Participants rated (on a scale of 0-5: 0 “no fatigue”—5 “extreme fatigue”) how much fatigue “they expected or imagined to feel immediately after completing each task/activity” for both the physical and mental subscales. Higher PFS scores indicate greater (i.e., more severe) perceived fatigability (range 0-50). PFS scores were imputed with sex-specific equations when 1-3 of the 10 items were missing (n=4 imputed PFS Physical scores, n=5 imputed PFS Mental scores) (Cooper R, Popham M, Santanasto A J, Hardy R, Glynn N W, Kuh D. Are BMI and inflammatory markers independently associated with physical fatigability in old age?Int J Obes. 2019; 43(4):832-41).

In addition, Perceived Exertion was measured at the second clinic visit using the Borg Rating of Perceived Exertion (RPE) Scale after a steady state walking task. Participants were instructed to walk on a treadmill for 5 minutes at a standard slow pace of 1.5 miles per hour (0.67 m/s) at 0% grade. After the task, participants were asked immediately to rate their perceived exertion using the Borg RPE scale (range 6-20; 6=no exertion at all, 9=very light, 11=light, 13=some-what hard, 20=maximal exertion) (13).

Performance Deterioration was assessed during the fast-paced 400 m walk. Performance Deterioration was defined as the percentage of lap time differences between 2^{nd} lap and the next to last (9^{th}) lap. Marked slowing was defined as an increase in lap time of ≥6.5% (Simonsick E M, Schrack J A, Glynn N W, Ferrucci L. Assessing fatigability in mobility-intact older adults. J Am Geriatr Soc. 2014 February; 62(2):347-51).

### Covariates

Age, sex, race (white/non-white), and smoking status (current/former/never) were obtained from self-administered questionnaires. Height (Harpenden stadiometers; Dyved UK) and weight (balance-beam scale) were measured by trained research staff. Multimorbidity (yes/no) included self-reported diabetes, heart diseases (including heart attack, coronary and myocardial infarction), stroke, lung diseases (including chronic obstructive lung disease, chronic bronchitis, asthma, emphysema, and chronic obstructive pulmonary disease), osteoarthritis and fall history during the past 12 months (including fallen and landed on the floor or ground or fallen and hit an object like a table or chair). Hypertension was defined by systolic blood pressure ≥130 mmHg and diastolic blood pressure 80 mmHg. All covariates were measured at the first clinic visit.

### Statistical Methods

Descriptive characteristics of participants were reported as mean±SD or frequencies (percentages) by better versus worse physical function (SPPB≥10 vs. SPPB<10), a key clinically relevant measure. Comparisons were examined using two-sample t tests for continuous variables and c² tests for categorical variables.

For the primary analyses, Pearson correlation coefficients (p) were used to assess univariate correlations of the PPFI against clinically relevant measures. Stratified analyses were conducted by sex and SPPB (10 vs. <10). For correlations between the PPFI and key clinically relevant variables showing significance (p<0.05), multivariable tobit regression, adjusted for age, sex, race, weight, height and smoking status were further performed, because PPFI was left censored at 0. Alpha was set to 0.05 and two-sided P values smaller than 0.05 were considered significant. All analyses were generated using Stata version 17 (Statacorp, College Station, TX), and all figures were generated using R (version 4.0).

### Example 2

### Results

### Participant Characteristics

Participants (N=64) had a mean age of 78.4 years, were approximately equally split between women (54.7%) and men, predominantly white (87.5%), and well-educated (82.8% had >high school education level). The majority of participants (n=47, 73.4%) had better physical function (SPPB≥10). Those with worse physical function were older (p=0.002), had slower chair stands speed (p<0.001), longer time to complete the fast-paced 400 m walk (p=0.036), lower leg peak power (p=0.005), and higher PFS Mental scores (p=0.031). No differences by physical function were found for height, weight, usual gait speed, time to complete the usual-paced 400 m walk, Performance Deterioration, PFS Physical scores, Perceived Exertion RPE fatigability, or prevalent diseases.

### Derivation of the Pittsburgh Performance Fatigability Index (PPFI)

Raw accelerometry data segmented from the 400 m walks were used for the two steps listed below to derive PPFI. The estimation of PPFI was performed in R (R Foundation for Statistical Computing, Vienna, Austria, version 4.0).

Step 1. Estimating stride and cadence:

Strides (i.e., a full step cycle) and cadence (i.e., rate of step cycles) for the fast- and usual-paced 400 m walks were estimated using the Adaptive Empirical Pattern Transformation (ADEPT) R package (Karas M, Stra Czkiewicz M, Fadel W, HarezlakJ, Crainiceanu C M, UrbanekJK. Adaptive empirical pattern transformation (ADEPT) with application to walking stride segmentation. Biostatistics. 2019 Sep. 23). ADEPT detects pattern repetitions in the observed data by maximizing local correlation between a scaled and translated pattern function and observed data. In this example case, for the scale parameters, a scale ranging from 0.8 to 1.6 seconds was used to represent the range of human cadence from 1.4 to 2.5 steps/second. A smoothing window length w=0.2 s was also used in the similarity maximization step and w=0.6 s was used to search for the local maxima. Strides were segmented by iteratively identifying local maxima of correlation function with five pre-defined empirical left-wrist-worn accelerometry stride templates provided in the ADEPT package and the observed data (Urbanek J K, Harezlak J, Glynn N W, Harris T, Crainiceanu C, Zipunnikov V. Stride variability measures derived from wrist- and hip-worn accelerometers. Gait Posture. 2017; 52:217-23). The raw cadence was estimated as steps/second and then used in Step 2 detailed below.

Step 2. Calculating PPFI:

The raw cadence estimates over the fast and usual pace 400 m walks were further smoothed to obtain individual-level smoothed cadence trajectories via penalized regression splines with five equally spaced knots and the smoothing parameter chosen by “REML” implemented in the “mgcv” R package (Ruppert D, Wand M P, Carroll R J. Semiparametric regression during 2003-2007. Electron J Stat. 2009 Jan. 1; 3:1193-256).

Based on the individual-level smoothed cadence trajectories, the area under the cadence-vs-time curve was obtained for each participant using “DescTools” R package with the default trapezoid method, and the maximum cadence for each participant was defined as the maximal (i.e., highest) value of cadence on the individual-level smooth trajectories during the 400 m walks. Conceptually, PPFI quantifies the percentage of performance decrement during the walking task by comparing area under the observed cadence-versus-time curve to a hypothetical area that would be observed in the absence of fatigue (i.e., if the participant sustained maximal cadence throughout the whole 400 m walk). Different weights were applied to two parts of the walking task in order to minimize the influence of a “motivation effect” (i.e., the participant speeds up) at the end of the walk (FIGS. 5A-5B).

The larger the PPFI, the greater the performance fatigability. Specifically, PPFI (FIGS. 6A-6B) is calculated as Equation (1):

$$[ ( 1 - \frac{A U {C}_{s t a r t \to t}}{{Cadence}_{max} \times t} ) * \frac{A U {C}_{s t a r t \to t}}{A U {C}_{s t a r t \to end}} + ( 1 - \frac{A U {C}_{t \to end}}{{Cadence}_{max} \times ( Total - t )} ) * \frac{A U {C}_{t \to end}}{A U {C}_{s t a rt \to end}} ] * 100 \% \qquad ( 1 )$$

where AUC_{start→t} represents the area under the cadence-vs-time from the beginning of the walk to t, AUC_{t→end} represents the area under the cadence-vs-time from t to the end of the walk, AUC_{start→end} represents the area under the cadence-vs-time during the entire walking task, cadenceₘₐₓ represents the maximum cadence identified during the entire walking task, Total represents the total time to complete the walking task (in seconds). For the fast-paced 400 m walk, t=300 seconds (i.e., 5 minutes); while for the usual-paced 400 m walk, t=360 seconds (i.e., 6 minutes). If Total <t, participant's PPFI was assigned as 0 because the previous studies have shown that participants completed fast-paced 400 m less than 5 mins had no performance deterioration (Simonsick E M, Newman A B, Visser M, Goodpaster B, Kritchevsky S B, Rubin S, et al. Mobility limitation in self-described well-functioning older adults: importance of endurance walk testing. J Gerontol A, Biol Sci Med Sci. 2008 August; 63(8):841-7) and they also had a gait speed>1.33 m/s, indicating better aerobic capacity and low fatigability (Alexander N B, Taffet G E, Horne F M, Eldadah B A, Ferrucci L, Nayfield S, et al. Bedside-to-Bench conference: research agenda for idiopathic fatigue and aging. J Am Geriatr Soc. 2010 May; 58(5):967-75).

Five minutes was chosen for the fast-paced walk and 6 minutes for the usual-paced walk in this study because it included the time that most participants needed to complete the 400 m walk, while still leaving on average at least 30 seconds at the end of the walk when participants usually sped up. The 5- and 6-minute time choices were selected due to generalizability with community-dwelling healthy older adults whose mean fast-paced gait speed typically ranges from 0.9-1.2 m/s and usual gait speed ranges from 0.6-1.0 m/s (Studenski S, Perera S, Patel K, Rosano C, Faulkner K, Inzitari M, et al. Gait speed and survival in older adults. JAMA. 2011 Jan. 5; 305(1):50-8).

Different weights were applied to the beginning part (first 5 or 6 minutes for fast- and usual-paced 400 m walks, respectively) and the ending part (remaining time to completion) of the walking task to: 1) minimize the influence of motivation effect at the end of the walk; and 2) emphasize that the performance decrement occurred at the beginning of the walk. The weights were individualized, proportional to the distance covered within the time t and beyond.

Alternatively, the two terms shown in Equation (1) can be re-expressed as ratios of the average cadence during each period divided by the maximum cadence. Using the definition of the average value of an integrable function on an interval in calculus as shown in Equations (2) and (3):

$$\frac{A U {c}_{s t a r t \to t}}{{Cadence}_{max} \times t} = \frac{Average {Cadence}_{start \to t} \times t}{{Cadence}_{max} \times t} = \frac{Average {Cadence}_{start \to t}}{{Cadence}_{max}} \qquad ( 2 )$$

$$\frac{{AUC}_{t \to end}}{{Cadence}_{max} \times ( Total - t )} = \frac{Average {Cadence}_{t \to end} \times ( Total - t )}{{Cadence}_{max} \times ( Total - t )} = \frac{Average {Cadence}_{t \to end}}{{Cadence}_{max}} \qquad ( 3 )$$

### Descriptive Characteristics of PPFI Scores

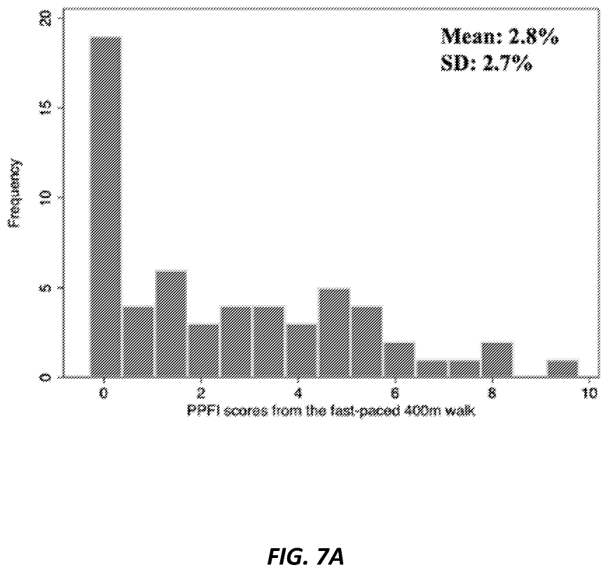

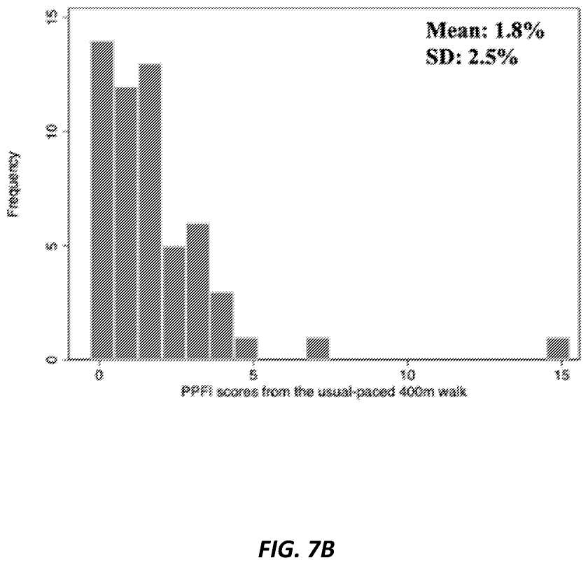

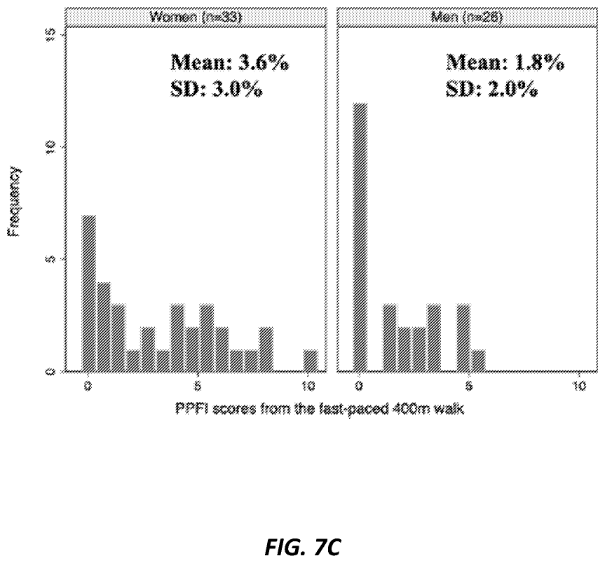

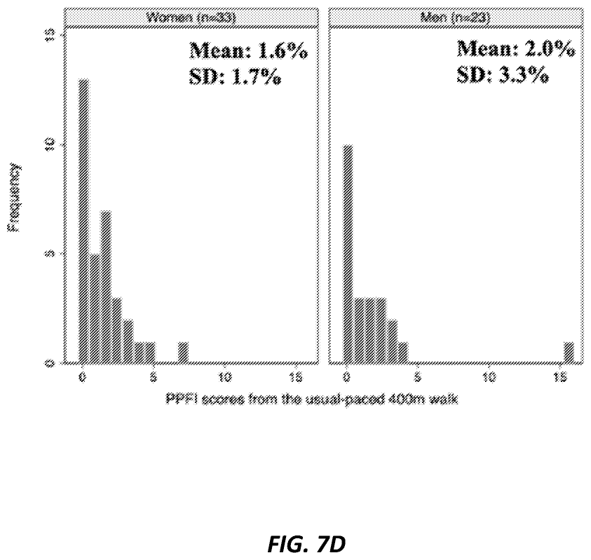

The distributions of the PPFI scores were right-skewed for both the fast- and usual-paced walks, yet the distribution of the PPFI scores from the fast-paced walk was spread more evenly across the distribution than for the usual-paced walk. Specifically, from the fast-paced 400 m walk (n=59), PPFI scores (i.e., percent of performance decrement) ranged from 0 to 10.1%, mean 2.8% [SD 2.7%](FIG. 7A). From the usual-paced 400 m walk (n=56), PPFI scores ranged from 0 to 15.5%, mean 1.8% [SD 2.5%](FIG. 7B). When stratified by sex, women had higher PPFI scores on average than men from the fast-paced walk (3.6%±3.0% for women, 1.8%±2.0% for men, P=0.015) (FIG. 7C), while women showed similar PPFI as men from the usual-paced walk (1.6%±1.7% for women, 2.0%±3.3% for men, P=0.54) (FIG. 7D). When stratified by SPPB, PPFI scores were similar between better (SPPB≥10) vs. worse (SPPB<10) physical function groups from both fast- or usual-paced walks (data not shown). In addition, among participants with a PPFI score calculated from both the fast- and usual-paced walk (n=51), Pearson correlation between the PPFI scores was 0.45 (P<0.001).

Associations of the PPFI Scores with Age and Clinically Relevant Measures

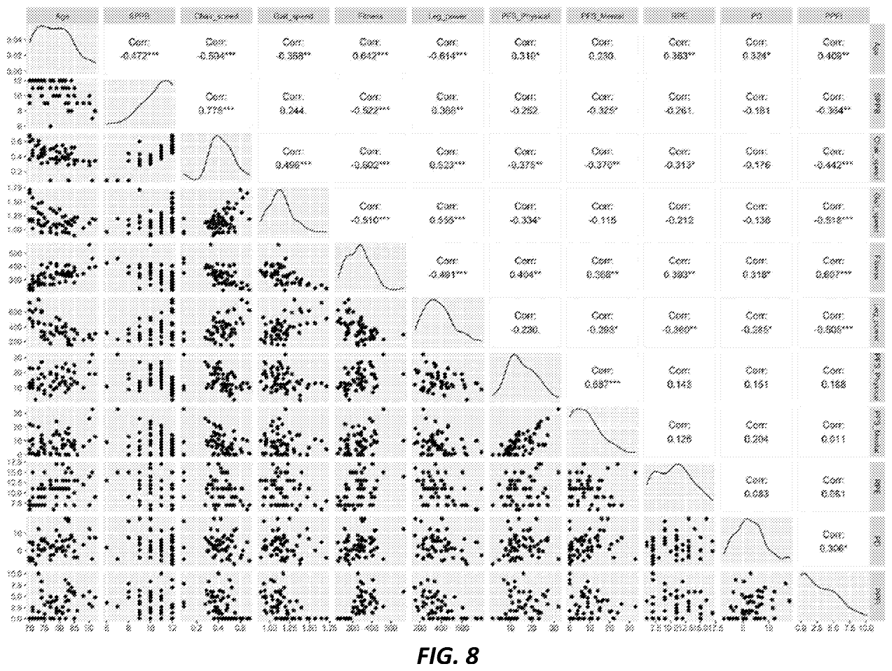

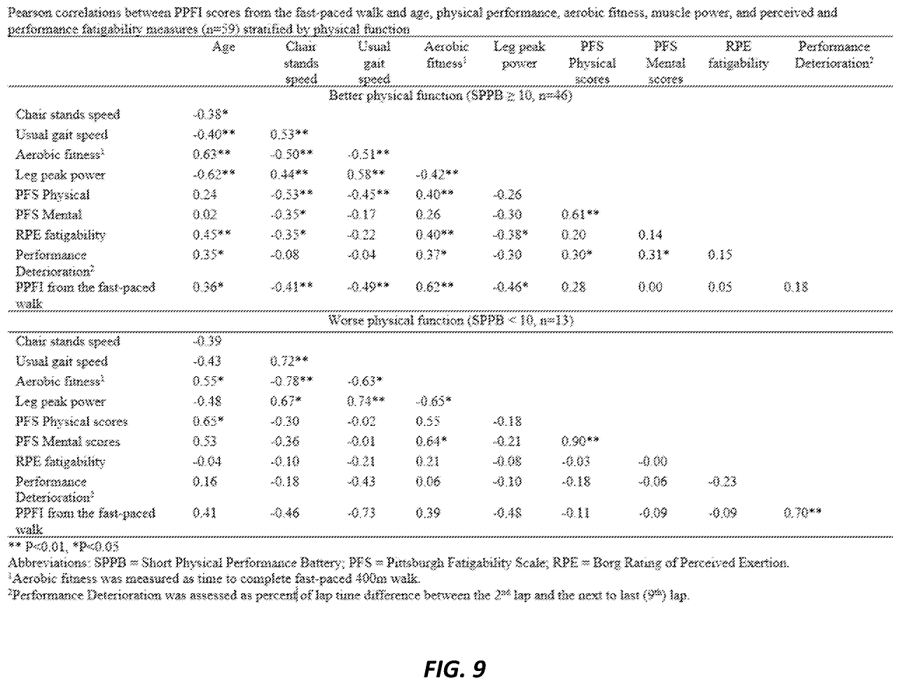

For the fast-paced walk, higher PPFI scores (i.e., greater performance fatigability) were strongly correlated with advanced age, worse SPPB, slower chair stands speed, slower usual gait speed, lower aerobic fitness, worse leg peak power, and greater Performance Deterioration (all p<0.05); PPFI scores were not correlated with PFS scores or Perceived Exertion RPE fatigability. Comparing the two performance fatigability measures (PPFI and Performance Deterioration, ρ=0.31), the PPFI showed stronger correlations against age and clinically relevant measures than Performance Deterioration (FIG. 8). When stratified by physical function, higher PPFI from the fast-paced walk among participants with better physical function (SPPB≥10, n=46) showed similar correlations with slower chair stands speed, slower usual gait speed, poorer physical fitness, and lower leg peak power compared to participants with worse physical function (SPPB<10, n=13) (FIG. 9). Notably, higher PPFI scores were more strongly correlated with greater Performance Deterioration among participants with worse physical function than among participants with better physical function, ρ=00.70 vs. 0.18, respectively (FIG. 9).

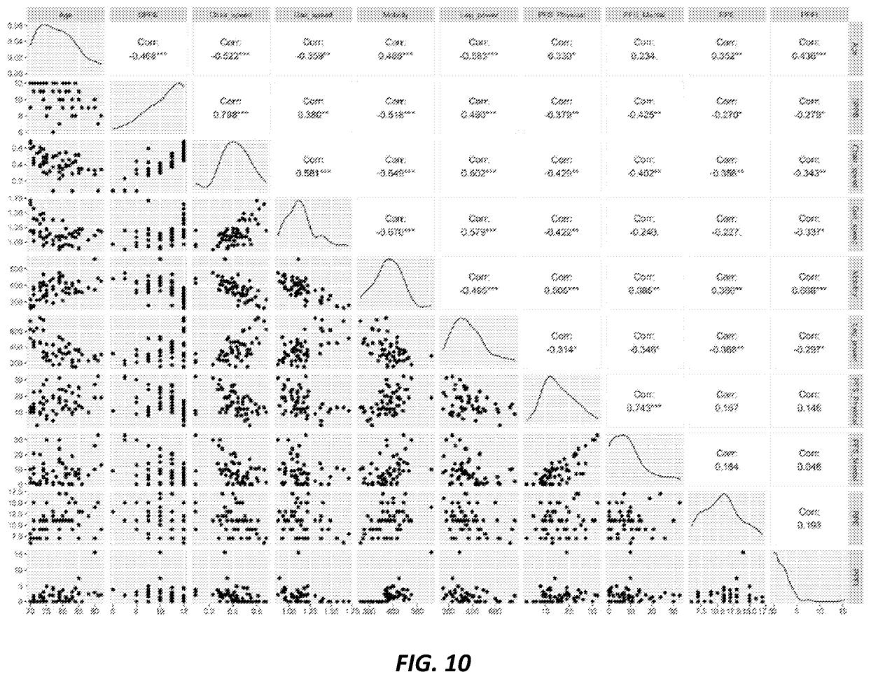

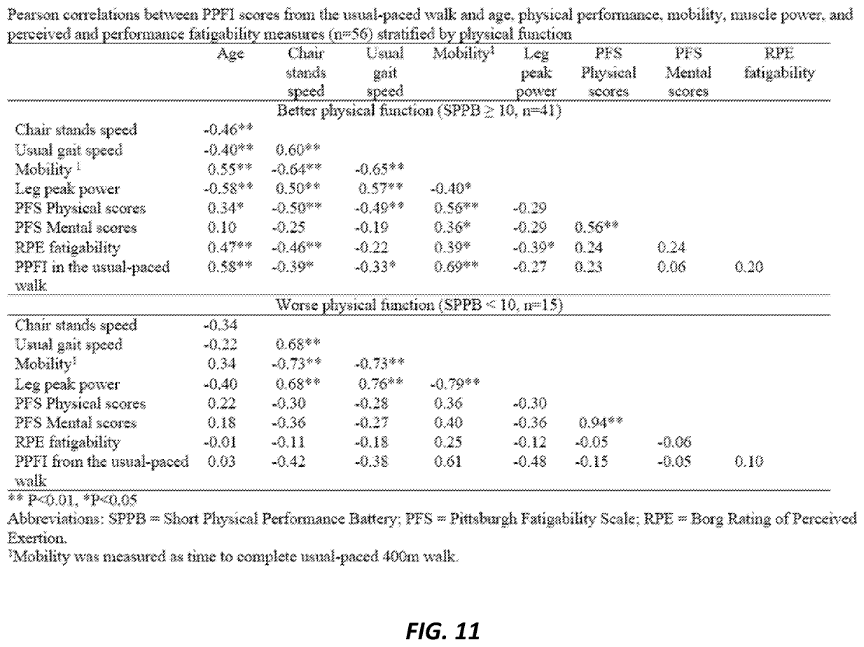

Comparably, for the usual-paced walk, higher PPFI scores were correlated, albeit moderately, with advanced age, worse SPPB, slower chair stands speed, slower usual gait speed, worse mobility, and worse leg peak power (FIG. 10). When stratified by SPPB, higher PPFI scores from the usual-paced walk among participants with worse physical function (SPPB<10, n=15) showed similar correlations with slower chair stands speed, slower usual gait speed, and worse mobility compared to participants with better physical function (SPPB≥10, n=41) (FIG. 11). However, higher PPFI scores were more strongly correlated with worse leg peak power among participants with worse physical function (FIG. 11).

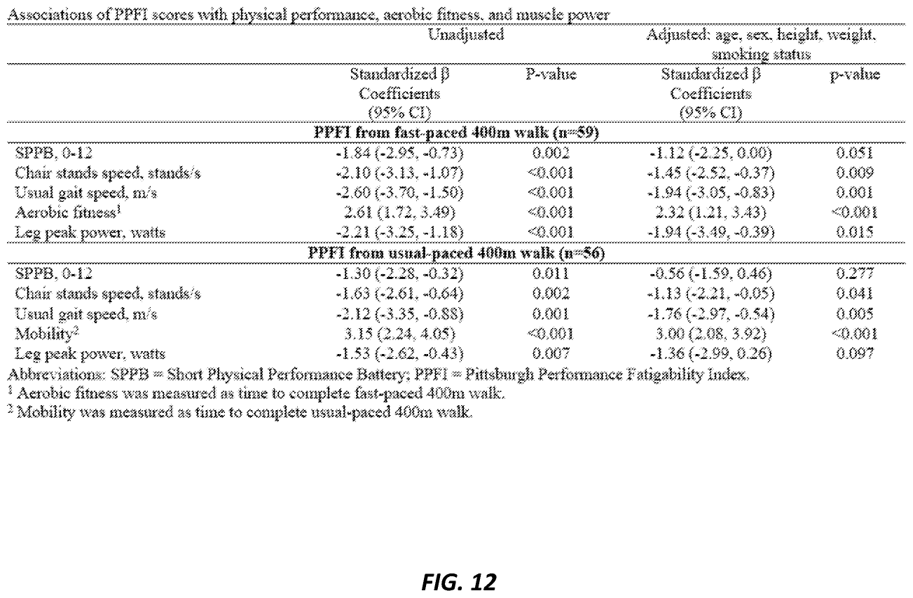

Higher PPFI scores from the fast-paced walk were significantly associated with slower chair stands speed (standardized b=−1.45, ρ=0.009), slower usual gait speed (standardized b=−1.94, ρ=0.001), worse aerobic fitness (standardized b=2.32, p<0.001), and lower leg peak power (standardized b=1.94, ρ=0.015), after adjustment for age, sex, height, weight, and smoking status (FIG. 12). However, there was no significant associations of PPFI scores with SPPB after covariate adjustment (FIG. 12).

Similarly, higher PPFI scores from the usual-paced walk were significantly associated with slower chair stands speed (standardized b=−1.13, ρ=0.041), slower usual gait speed (standardized b=−1.76, ρ=0.005), and worse mobility (standardized b=3.00, p<0.001), after adjustment for age, sex, height, weight, and smoking status (FIG. 12). However, there was no significant association of PPFI scores with SPPB and leg peak power after covariate adjustment (FIG. 12).

### Example 3

### Discussion

This study introduced a measure—the Pittsburgh Performance Fatigability Index (PPFI)—to quantify walking-based performance fatigability using wrist-worn accelerometry among older adults and summarized the characteristics of PPFI score and its associations with age, physical performance, muscle power and other fatigability measures. Overall, PPFI scores from the fast-paced walk were higher and more variable than from the usual-paced walk and revealed sex differences. Higher PPFI scores from the fast-paced walk were strongly negatively correlated with all physical function measures and leg peak power, while PPFI scores from the usual-paced walk were moderately negatively correlated with these measures, which all predict health decline (Lindemann U, Krumpoch S, Becker C, Sieber C C, Freiberger E. The course of gait speed during a 400 m walk test of mobility limitations in community-dwelling older adults. Z Gerontol Geriatr. 2021 Jun. 11; Guralnik J M, Simonsick E M, Ferrucci L, Glynn R J, Berkman L F, Blazer D G, et al. A short physical performance battery assessing lower extremity function: Association with self-reported disability and prediction of mortality and nursing home admission. J Gerontol. 1994 March; 49(2):M85-94; Studenski S, Perera S, Patel K, Rosano C, Faulkner K, Inzitari M, et al. Gait speed and survival in older adults. JAMA. 2011 Jan. 5; 305(1):50-8; Peel N M, Kuys S S, Klein K. Gait speed as a measure in geriatric assessment in clinical settings: a systematic review. J Gerontol A, Biol Sci Med Sci. 2013 January; 68(1):39-46). After covariate adjustment, PPFI scores from the fast-paced walk were still significantly associated with all physical function measures (except SPPB scores) and leg peak power, whereas PPFI scores from the usual-paced walk were significantly associated with physical function measures (except SPPB scores) but not leg peak power. Collectively, the findings illustrate the validity and the utility of an objective and sensitive accelerometry-based performance fatigability measure for older adults.

To overcome constraints inherent in existing performance fatigability measures, the PPFI was developed to objectively detect performance decrement during a standardized task (e.g., fixed intensity and fixed distance). PPFI evaluates the “degree” or “severity” of decrement in walking performance during the walking task, strongly aligning with the definition of performance fatigability. PPFI scores have a finite range from 0 to 100, which allows standardized comparisons across different walking tasks. Additionally, PPFI does not assume that cadence declines constantly or linearly during the walking task, which overcomes one of the limitations of the existing measures. Importantly, the PPFI utilizes the whole spectrum of cadence trajectory during the walking task to locate the maximal cadence. It also reflects one's failure to keep up with or maintain their maximum cadence. Additionally, individualized weights were applied to minimize the influence of motivation effect while emphasizing the performance decrement that may occurred at the beginning of the walk. Furthermore, PPFI can be applied to different paced fixed-long-distance walks, although participants tend to show a lesser degree of performance fatigability during the usual-paced walk as shown in this study.

PPFI proved to be a more sensitive measure of performance fatigability than Performance Deterioration in this study, evidenced by the stronger correlations between PPFI scores and clinically relevant measures (FIG. 8). The magnitude of these correlations were similar when stratified by physical function and were comparable with previous studies (Schnelle J F, Buchowski M S, Ikizler T A, Durkin D W, Beuscher L, Simmons S F. Evaluation of two fatigability severity measures in elderly adults. J Am Geriatr Soc. 2012 Aug. 2; 60(8):1527-33; Simonsick E M, Schrack J A, Glynn N W, Ferrucci L. Assessing fatigability in mobility-intact older adults. J Am Geriatr Soc. 2014 February; 62(2):347-51; Vestergaard S, Patel K V, Bandinelli S, Ferrucci L, GuralnikJM. Characteristics of 400-meter walk test performance and subsequent mortality in older adults. Rejuvenation Res. 2009 June; 12(3):177-84). For instance, the performance fatigability severity, assessed as percent change in walking speed within the first 2.5 minute interval to the walking speed over the entire distance a participant walked during a usual-paced 10-minute walk, showed similar correlation with usual gait speed (p=−0.54) (Schnelle J F, Buchowski M S, Ikizler T A, Durkin D W, Beuscher L, Simmons S F. Evaluation of two fatigability severity measures in elderly adults. J Am Geriatr Soc. 2012 Aug. 2; 60(8):1527-33) as observed in the study (p=−0.52 for PPFI scores from the fast-paced walk and p=−0.34 for PPFI scores from the usual-paced walk). These similar correlations are not too surprising given that performance fatigability severity compares early, hence ideally maximum speed to overall average speed while the PFFI compares maximum cadence to average cadence, with the PPFI “screening” the whole cadence trajectory to find the maximum cadence and additionally giving more weight to the core of the walk while accounting for the “motivation effect” by giving less weight at the end the walk where participants tend to speed up. These clinically relevant measures predict health status, disability, and mortality (Pavasini R, Guralnik J, Brown J C, di Bari M, Cesari M, Landi F, et al. Short Physical Performance Battery and all-cause mortality: systematic review and meta-analysis. BMC Med. 2016 Dec. 22; 14(1):215; Peel N M, Kuys S S, Klein K. Gait speed as a measure in geriatric assessment in clinical settings: a systematic review. J Gerontol A, Biol Sci Med Sci. 2013 January; 68(1):39-46). Strong correlations between PPFI scores and these measures demonstrate that the PPFI is sensitive to physical and muscle factors that can reflect disability. Therefore, PPFI is measuring the construct of performance fatigability with great sensitivity.

As described above, the study derived performance fatigability from a usual-paced 400 m walk. Yet, unlike PPFI scores from the fast-paced walk, the correlations between PPFI scores from the usual-paced walk with clinically relevant measures were moderate. One potential explanation is that older adults are more likely to experience fatigue during a more strenuous walking task (Alexander N B, Taffet G E, Horne F M, Eldadah B A, Ferrucci L, Nayfield S, et al. Bedside-to-Bench conference: research agenda for idiopathic fatigue and aging. J Am Geriatr Soc. 2010 May; 58(5):967-75), such as a fast-paced walk, whereas they tend to self-pace during a usual-paced walk (Eldadah B A. Fatigue and fatigability in older adults. PM R. 2010 May; 2(5):406-13). Self-pacing would more likely maintain a relatively constant cadence and not exhibit a pronounced maximum cadence. Therefore, performance fatigability can be task specific (Hunter S K. Performance fatigability: mechanisms and task specificity. Cold Spring Harb Perspect Med. 2018 Jul. 2; 8(7)), and more easily to exhibit in long-distance fast-paced walking tasks than usual-paced walking tasks. Additionally, sex difference of PPFI scores were observed from the fast-paced walk, but not from the usual-paced walk, signaling that sex differences in factors relevant to fatigability, such as skeletal muscle physiology, muscle perfusion and voluntary activation, could be specific to task demands (Hunter S K. The relevance of sex differences in performance fatigability. Med Sci Sports Exerc. 2016 November; 48(11):2247-56).

Interestingly, PPFI scores from the fast-paced walk showed a significant association with leg peak power in the overall sample after adjustment, while PPFI scores from the usual-paced walk did not. This finding is plausible because a faster than usual-paced walk that requires sustainment for 400 m invokes leg power for the push-off phase, and thus may be a more sensitive indicator of deficient leg power (Foulis S A, Jones S L, van Emmerik R E, Kent J A. Post-fatigue recovery of power, postural control and physical function in older women. PLoS One. 2017 Sep. 7; 12(9):e0183483). Furthermore, in the study, older adults with worse physical function had a larger and significant correlation between PPFI from the usual-paced walk and leg peak power than those with better physical function, suggesting that older adults with worse physical function might have already utilized their maximal power capacity to finish the task at their usual pace, potentially due to lower physiological reserves (Foulis S A, Jones S L, van Emmerik R E, Kent J A. Post-fatigue recovery of power, postural control and physical function in older women. PLoS One. 2017 Sep. 7; 12(9):e0183483), and high disease burden (Lange-Maia B S, Newman A B, Strotmeyer E S, Harris T B, Caserotti P, Glynn N W. Performance on fast- and usual-paced 400-m walk tests in older adults: are they comparable?Aging Clin Exp Res. 2015 June; 27(3):309-14).

Although a few studies have found strong correlations between performance fatigability and perceived fatigability (Schnelle J F, Buchowski M S, Ikizler T A, Durkin D W, Beuscher L, Simmons S F. Evaluation of two fatigability severity measures in elderly adults. J Am Geriatr Soc. 2012 Aug. 2; 60(8):1527-33; Simonsick E M, Schrack J A, Glynn N W, Ferrucci L. Assessing fatigability in mobility-intact older adults. J Am Geriatr Soc. 2014 February; 62(2):347-51; Glynn N W, Santanasto A J, Simonsick E M, Boudreau R M, Beach S R, Schulz R, et al. The Pittsburgh Fatigability Scale for older adults: development and validation. J Am Geriatr Soc. 2015 January; 63(1):130-5), we did not find PPFI scores correlated with perceived fatigability. In previous papers where the associations were found (Schnelle J F, Buchowski M S, Ikizler T A, Durkin D W, Beuscher L, Simmons S F. Evaluation of two fatigability severity measures in elderly adults. J Am Geriatr Soc. 2012 Aug. 2; 60(8):1527-33; Simonsick E M, Schrack J A, Glynn N W, Ferrucci L. Assessing fatigability in mobility-intact older adults. J Am Geriatr Soc. 2014 February; 62(2):347-51), perceived fatigability was measured after completing the same walking task as the performance fatigability measure, whereas perceived fatigability in this study was measured by questionnaire with the PFS and perceived exertion after a treadmill-based slow walking task. The PFS covers a wide span of activity from sedentary to moderate and high-intensity, and also includes social activities (Glynn N W, Santanasto A J, Simonsick E M, Boudreau R M, Beach S R, Schulz R, et al. The Pittsburgh Fatigability Scale for older adults: development and validation. J Am Geriatr Soc. 2015 January; 63(1):130-5). Thus, it is reasonable to assume that walking-based performance fatigability might only represent a sub-category of fatigability that is measured by the PFS. As for Perceived Exertion RPE fatigability, its correlation with PPFI scores was also poor likely due to the slow walking task that may not be strenuous enough to produce fatigue among our higher functioning study population.

Furthermore, previous work showed that despite a moderate correlation between Perceived Exertion RPE fatigability and Performance Deterioration, the two measures actually classified different participants with greater fatigability (Simonsick E M, Schrack J A, Glynn N W, Ferrucci L. Assessing fatigability in mobility-intact older adults. J Am Geriatr Soc. 2014 February; 62(2):347-51). Similarly, studies among multiple sclerosis (Loy B D, Taylor R L, Fling B W, Horak F B. Relationship between perceived fatigue and performance fatigability in people with multiple sclerosis: A systematic review and meta-analysis. J Psychosom Res. 2017 Jun. 27; 100:1-7; Seamon B A, Harris-Love M O. Clinical Assessment of Fatigability in Multiple Sclerosis: A Shift from Perception to Performance. Front Neurol. 2016 Nov. 7; 7:194; Enoka R M, Almuklass A M, Alenazy M, Alvarez E, Duchateau J. Distinguishing between Fatigue and Fatigability in Multiple Sclerosis. Neurorehabil Neural Repair. 2021 Sep. 28; 15459683211046256) and rheumatoid arthritis (Marrelli K, Cheng A J, Brophy J D, Power G A. Perceived versus performance fatigability in patients with rheumatoid arthritis. Front Physiol. 2018 Oct. 10; 9:1395) patients indicate that performance fatigability and perceived fatigability do not assess the same construct and should be evaluated independently.

The cross-sectional study described herein had several strengths. First, the study included both men and women, with more than half of the sample ≥80 years. Yet, study participants were relatively well-educated and heathier than the general population of older adults. This study evaluated multiple fatigability measures and implemented both fast- and usual-paced 400 m walks, which is seldom available in a single study. Second, personalized weights were applied based on percentage of distance covered within time t and beyond, which helps to minimize influence of motivation effects while still utilizing data from the full walk to derive PPFI. The choice of exact time cutoff points may differ for populations with different characteristics. For populations with multi-morbidities, the 5 minutes cutpoint may be replaced with an appropriate percentile of sample completion time. For example, in this study, 5 minutes equaled the 25^{th} percentile of the sample's fast-paced 400 m completion time. Lastly, manually segmenting raw accelerometry data corresponding to the walking tasks is relatively time-consuming.

In conclusion, the PPFI is a valid, objective and sensitive measure of performance fatigability during fast- and usual-paced walks among older adults. PPFI captures undetectable performance fatigability improving upon existing performance fatigability measures.

### Example 4

Classification and regression tree (CART) models using the “rpart” R package were applied to generate cut-point(s) in PPFI scores that most strongly discriminated gait speed. Participants with PPFI=0 were grouped as their own individual group as they showed no performance fatigability during the walking task. Then, the remainder of the sample was split into training and testing sets (80% versus 20%). Using the training set, trees were built using analysis of variance (ANOVA) with at least 10 observations in a node and cross-validation was performed by randomly partitioning the data into 10 equally sized mutually exclusive data sets. The final tree was pruned to the most parsimonious tree that was within 1 standard error (SE) of the tree with the smallest prediction error (mean square error). Then, the predictions were visually examined with the optimally pruned tree using the testing set. Lastly, to examine the discriminant power of the identified PPFI cut-points, linear regressions were conducted to obtain least squared means±standard errors of physical function, leg peak power and VO₂peak, adjusted for age, sex, race, height, weight, and smoking status. All analyses were repeated with stratification by sex to evaluate if sex-specific cut-points were warranted for performance fatigability severity measured with PPFI.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method comprising:
receiving raw accelerometry data, the raw accelerometry data being collected from a subject during a walking task; and
analyzing the raw accelerometry data to calculate a performance fatigability index for the subject.

2. The method of claim 1, wherein the performance fatigability index for the subject represents a percentage of decrement in the subject's performance during the walking task.

3. The method of claim 2, wherein the performance fatigability index for the subject comprises a comparison between an area under an observed gait cadence-versus-time trajectory curve for the walking task and an area under a hypothetical gait cadence-versus-time trajectory curve for a walking task at maximal cadence.

4. The method of claim 1, wherein the step of analyzing the raw accelerometry data comprises obtaining an area under an observed gait cadence-versus-time trajectory curve for the walking task, wherein the performance fatigability index for the subject is calculated using the area under the observed gait cadence-versus-time trajectory curve for the walking task.

5. The method of claim 4, wherein the step of obtaining the area under the observed gait cadence-versus-time trajectory curve for the walking task comprises:
estimating a plurality of strides and a plurality of cadences for the walking task based on the raw accelerometry data;
smoothing the cadences for the walking task to obtain a smoothed cadence trajectory; and
determining the area under the observed gait cadence-versus-time trajectory curve for the walking task based on the smoothed cadence trajectory.

6. The method of claim 1, wherein the step of analyzing the raw accelerometry data comprises:
partitioning the walking task into a plurality of segments;
obtaining a first area under an observed gait cadence-versus-time trajectory curve for a first segment of the walking task;
obtaining a second area under the observed gait cadence-versus-time trajectory curve for a second segment of the walking task; and
assigning respective weights to each of the first and second areas under the observed gait cadence-versus-time trajectory curve, wherein the performance fatigability index for the subject is calculated using a sum of the weighted first and second areas under the observed gait cadence-versus-time trajectory curve.

7. The method of claim 1, further comprising collecting the raw accelerometry data from the subject while the subject completes the walking task.

8. The method of claim 1, wherein the raw accelerometry data is received from a triaxial accelerometer.

9. The method of claim 8, wherein the triaxial accelerometer is worn by the subject.

10. The method of claim 1, wherein a length of the walking task is selected to elicit a change in the subject's cadence during the walking task.

11. The method of claim 10, wherein the length of the walking task is at least 400 meters.

12. The method of claim 10, wherein the length of the walking task is at least 4 minutes.

13. The method of claim 1, wherein the walking task is a walk at a usual pace for the subject.

14. The method of claim 1, wherein the walking task is a walk at a fast pace for the subject.

15. The method of claim 1, further comprising classifying the subject into one of a plurality of physical performance decline categories based on the performance fatigability index.

16. A system comprising:
a triaxial accelerometer; and
a computing device operably coupled to the triaxial accelerometer, the computing device comprising a processor and a memory operably coupled to the processor, the memory having computer-executable instructions stored thereon that, when executed by the processor, cause the processor to:
receive raw accelerometry data from the triaxial accelerometer, the raw accelerometry data being collected from a subject during a walking task; and
analyze the raw accelerometry data to calculate a performance fatigability index for the subject.

17. The system of claim 16, wherein the performance fatigability index for the subject represents a percentage of decrement in the subject's performance during the walking task.

18. The system of claim 17, wherein the performance fatigability index for the subject comprises a comparison between an area under an observed gait cadence-versus-time trajectory curve for the walking task and an area under a hypothetical gait cadence-versus-time trajectory curve for a walking task at maximal cadence.

19. The system of claim 16, wherein the step of analyzing the raw accelerometry data comprises obtaining an area under an observed gait cadence-versus-time trajectory curve for the walking task, wherein the performance fatigability index for the subject is calculated using the area under the observed gait cadence-versus-time trajectory curve for the walking task.

20. The system of claim 19, wherein the step of obtaining the area under the observed gait cadence-versus-time trajectory curve for the walking task comprises:
estimating a plurality of strides and a plurality of cadences for the walking task based on the raw accelerometry data;
smoothing the cadences for the walking task to obtain a smoothed cadence trajectory; and
determining the area under the observed gait cadence-versus-time trajectory curve for the walking task based on the smoothed cadence trajectory.

21. The system of claim 16, wherein the step of analyzing the raw accelerometry data comprises:
partitioning the walking task into a plurality of segments;
obtaining a first area under an observed gait cadence-versus-time trajectory curve for a first segment of the walking task;
obtaining a second area under the observed gait cadence-versus-time trajectory curve for a second segment of the walking task; and
assigning respective weights to each of the first and second areas under the observed gait cadence-versus-time trajectory curve, wherein the performance fatigability index for the subject is calculated using a sum of the weighted first and second areas under the observed gait cadence-versus-time trajectory curve.

22. The system of claim 16, wherein a length of the walking task is selected to elicit a change in the subject's cadence during the walking task.

23. The system of claim 22, wherein the length of the walking task is at least 400 meters.

24. The system of claim 22, wherein the length of the walking task is at least 4 minutes.

25. The system of claim 16, wherein the walking task is a walk at a usual pace for the subject.

26. The system of claim 16, wherein the walking task is a walk at a fast pace for the subject.

27. The system of claim 16, wherein the memory has further computer-executable instructions stored thereon that, when executed by the processor, cause the processor to classify the subject into one of a plurality of physical performance decline categories based on the performance fatigability index.
